(19) United States
(12) Patent Application Publication
(10) Pub. No.: US 2022/0241510 A1
(43) Pub. Date: Aug. 4, 2022

(54) Medical Syringe with a Thermostat

(71) Applicant: LESLIE NESBITT, New York, NY (US)

(72) Inventor: LESLIE NESBITT, New York, NY (US)

(21) Appl. No.: 17/167,421

(22) Filed: Feb. 4, 2021

(51) Int. Cl. A61M 5/315; A61M 5/46; A61M 5/24

(52) U.S. Cl. CPC A61M 5/31533; A61M 2205/3368; A61M 5/24; A61M 5/46

(57) ABSTRACT

The invention relates to a medical syringe device with thermostat, for a medical instrument, to insert a needle into a. patient's body to a determined insertion depth. The safety medical syringe device, comprises a hub, a needle, a protecting shield, longitudinal resilient means, transverse resilient means, a housing, and is provided with different technical means configured to cooperate with each other in order to perform different functions of the device. The proposed device enables to control the temperature of the device, wherein the device is a disposable one and it ensures its user an injection as per medical requirements.

### BACKGROUND

### Field of the Invention

The present Invention generally relates to medical devices and its main function is to provide health care and medical care in such a safe manner which provides thermostat to measure the temperature of its contents.

### Description of the Related Art

In recent years, there has been a substantial increase in the use of individually sterilized, individually packaged so-called one-time use medical syringes. They gained popularity and the appreciation of medical personnel and also generally decreased the possibility of infecting an injected patient with germs or viruses that sometimes survived the sterilization process, a situation which occurred when multiple use syringes were sterilized after each use.

So-called one-time use medical syringes, presently dominating today's market, also have many other well-known advantages and disadvantages. There are multiple inventions that have been seen in prior art regarding advancements in similar field. For instance, a Medical syringe filling and valving bearing U.S. Pat. No. 8,992,505B2 is issued to Thorne Consulting and Intellectual Property LLC. The patent implies apparatus and methods for providing a field environment filled proximal chamber of a multi-chamber syringe and for filling a prime-free syringe are disclosed. In addition, methods for resetting valves of a multi-chamber syringe without special tools or insertion of foreign objects into the syringe are disclosed. A valve for interfacing with syringes telescopically inserted into a larger syringe and which are generally used as proximal chambers in multi-chamber syringes used in mixing and serial delivery applications is disclosed. Four embodiments of novel resettable valves are disclosed. Also, a syringe assembly for delivering a saline-dose-saline medication sequence is disclosed.

Another patent on One-time use medical syringe invention bearing U.S. Pat. No. 4,687,467A is issued to Bozena Cygielski. The patent is on medical care device for only one-time use as a medical syringe for injection with destroyable piston after single use. Destruction of a syringe piston will occur after one-time use by cutting out a hole in the center of the front wall of the piston. In the very last moment of the injection stroke, the piston is pushed against the very sharp cylindrical cutting edge of the rear end of the tubular needle, which protrudes to the inside of the syringe cylinder for this purpose. With such a punctured piston it will be impossible to create a vacuum suction effect which is necessary to introduce any medication or liquid into the syringe cylinder for a second use. Simply put, nobody will be able to use this kind of syringe twice.

A Medical syringe with safety shield system bearing US patent 2,005,0159706A1 is issued to Becton Dickinson and Co. The patent is on a medical device for delivering a medicament to a patient includes a syringe assembly having a barrel defining a reservoir containing the medicament, a needle cannula coupled to a forward end of the barrel, and a plunger having a stopper positioned in the barrel and movable into the barrel to cause the medicament to be expelled. The medical device also includes a cap arranged on, and slidable over, the needle cannula from a first position in which the forward tip of the needle cannula is exposed, to a second position in which the forward tip of the needle cannula is covered by the cap. An actuation mechanism connected to the cap includes an urging member coupled to the barrel and the cap for urging the cap toward the second position. A trigger element releasably secures the urging member in a charged state and releasably secures the cap in the first position. The trigger element is actuatable to release the cap by either manual actuation or by interaction with the thumb pad.

Another patent on Disposable safety medical syringe bearing U.S. Pat. No. 4,955,868A is issued to Edward Klein. The patent is on modification of a hypodermic syringe shielding the needle from user so that inadvertant puncture to the user of syringe will not occur before, during or after the injection of subject. This modification removes the necessity for capping or recapping the syringe. It is an improvement over prior devices in that the hypodermic needle is moved out of shield with out pressure of the syringe on subject being injected allowing for full view of needle during injection making venipuncture possible. The needle retracts automatically back in shield with release of pressure on shield handle (5) and syringe handle (7) by means of spring (2). Gasket (3) prevents movement of needle in shield by unintentional forces, and the plunger rod handle operates independently of retracting or protracting needle allowing for filling of syringe, aspiration of syringe and injecting with syringe. This device allows the safe use of a medical hypodermic syringe with out fear of contracting diseases the subject being injected may have by inadvertant puncture of user afer injecting subject. It also gives operator full visability of needle, control of needle and indendent action of exposing and needle and injecting with syringe or withdrawing blood of patient through springe.

A patent on Medical syringe holding/transport apparatus bearing U.S. Pat. No. 5,823,363A is issued to Douglas Cassel. The patent is on an article of manufacture which is constructed to receive and hold a plurality of syringes of differing size. The article comprises a substantially rigid frame having a plurality of syringe-receiving cavities formed therein. Each of the syringe-receiving cavities is sized and configured to receive and hold a syringe in a generally vertical orientation. At least some of the syringe-receiving cavities comprise variable diameter syringe-receiving cavities which are adapted to receive different sizes of syringes.

Another patent on Medical syringe bearing U.S. Pat. No. 4,553,962A is issued to Jean-Louis Brunet. The patent is on a medical syringe of the type in which an ampule and needle are received in the syringe barrel and a plunger is triggered to drive the needle from the barrel has an ampule whose body is composed of one material, preferably glass, while the needle is sealed and guided by a disk or plug of a different material.

Another patent on Medical syringe U.S. Pat. No. 4,650,468A is issued to Baldwin P. Jennings, Jr. The patent is on a single use disposable medical syringe is constructed and operated in a manner which prevents transmission of infection by the syringe after its use for inoculation. The syringe is marketed in a sterile package ready for use. The syringe needle is then covered by a shield. The syringe plunger assembly is then releasably locked to the barrel of the syringe. Following opening of the sterile package and removal of the needle shield, the plunger assembly is operated to fill the syringe barrel with medication and to inject the patient. Following injection, a piston head housing is unlocked from the syringe barrel and the plunger assembly is retracted toward the rear of the barrel and becomes automatically locked in this position with the piston head housing. The needle is disposed within the barrel rearwardly of a safety shield on the forward end of the barrel containing a safety membrane.

There are multiple solutions that have been presented in prior art. However, these solutions are limited and restricted to their conventional architecture, installation system and have considerable shortcomings which adversely affect the convenience with which they can be used. The prior systems have certain limitations including the design of assemblies which make them not suitable for every type of user. Moreover, the ease-of-use ability of these assemblies is also questionable.

It has to be noted that the current invention proposes an assembly with an advancement where the proposed assembly provides a medical syringe with a thermostat that can determine the temperature of its content.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

in light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole,

The primary desirable object of the present invention is to provide a novel and improved form of an injection with thermostat to measure temperature of contents within the syringe.

Another object of the present invention is to provide a method of incorporating a thermostat within the syringe assembly for effective dosage administration.

Another object of this invention is to provide a new and improved assembly which is designed with thermostat which is automatically activated. once the medicament dosage is in the syringe.

It is an object of the invention in one of its aspects to provide attributes are that such syringe assemblies require no change in current medicament delivery techniques, allow for dose adjustment, are preferably autoclavable, and allow for the inspection of contents.

Yet another object of the present invention is to provide a structure that is relatively inexpensive to produce so that ownership is available to a substantial majority of small and medium size businesses.

It is also the objective of the invention to provide a smart assembly which provide ease of use and convenience to everyone.

It is a further aspect of the present invention to provide a new and improved assembly which is of a durable and reliable construction and can sustain its effectiveness after multiple and consistent use.

Thus, it is the objective to provide a new and improved medical syringe apparatus with thermostat. Other aspects, advantages and novel features of the present invention will become apparent from the detailed description of the invention when considered in conjunction with the accompanying drawings.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 discloses the packaging box as per preferred embodiments of the invention.

FIG. 2 discloses view of proposed assembly as per additional embodiments of the invention.

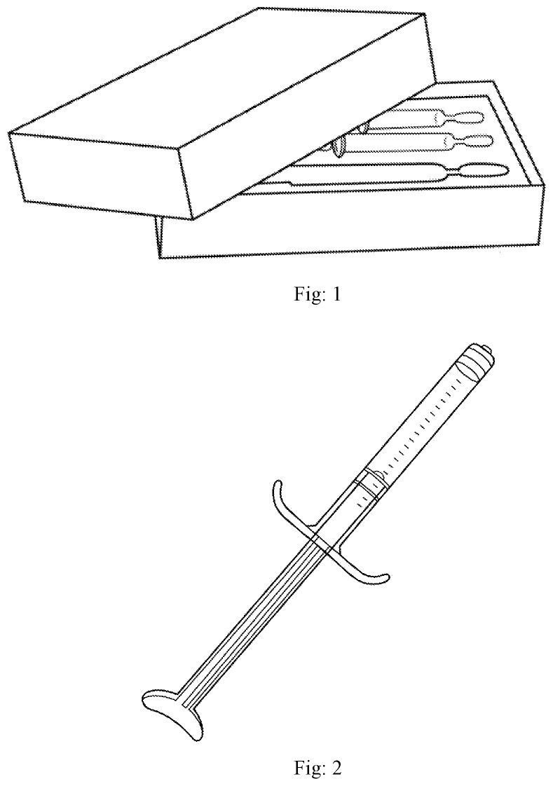

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention is directed to provide an improved medical syringe with thermostat to monitor the temperature of ingredients within the syringe.

Advancements to polymers and injection molding technologies have contributed to widespread adoption of plastics in the medical industry. All segments of the medical industry have embraced the benefits of plastics, and are looking to incorporate more plastic in their designs in 2018 and beyond. Low-cost plastics, for example, have enabled the creation of disposable plastic instruments, syringes, gloves and gowns that, in turn, help reduce the spread of infections within the hospital environment. Plastics have also replaced glass in a variety of laboratory consumables, improving lab safety while reducing consumable costs.

Accordingly, in one aspect of the present invention, the disposable injection device with thermostat in combination comprises:

A housing containing a non-exchangeable drug-cartridge for storing a liquid drug sufficient for a number of injections.

A settable dose setting mechanism having a dose setting button whereby a user can set or select a random size of a dose to be injected, A telescopic needle covering shield, which shield distally carries a cleaner and is operational between a first position and a second position;

The first position defined as a position in which the telescopic shield is in an extended position covering a tip of a front-part of the needle cannula,

The second position defined as a position in which the telescopic shield is retracted such that at least the tip of the front-part of the needle cannula is exposed to perform an injection.

The lumen of the needle cannula is preserved in a sterile condition prior to the initial injection and resilient means are provided for automatically returning the shield to its first position following both the initial injection and any of the subsequent injections. Further, the tip of the needle cannula is maintained inside the cleaner between injections.

Since the injection device is delivered ready to use in a sterile condition, the initial injection is always performed with a sterile needle with thermostat which monitors the temperature of liquid dose.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A medical syringe with thermostat assembly, comprising:
A housing containing a cartridge for storing a liquid drug for a number of injections,
A settable dose setting mechanism having a dose whereby a user can set a size of a dose to be injected,
A thermostat assembly within the injection to monitor the temperature of liquid to be injected to user.
